(19) United States
(12) Patent Application Publication
(10) Pub. No.: US 2020/0197744 A1
(43) Pub. Date: Jun. 25, 2020

(54) METHOD AND SYSTEM FOR MOTION MEASUREMENT AND REHABILITATION

(71) Applicant: MOTION SCIENTIFIC INC., Santa Ana, CA (US)

(72) Inventor: NICOLAS SCHWEIGHOFER, Santa Monica, CA (US)

(73) Assignee: MOTION SCIENTIFIC INC., SANTA ANA, CA (US)

(21) Appl. No.: 16/722,416

(22) Filed: Dec. 20, 2019

Related U.S. Application Data
(60) Provisional application No. 62/784,262, filed on Dec. 21, 2018.

(51) Int. Cl. A63B 21/00; A61B 5/11; G06T 7/277; G06T 13/40; G06K 9/00

(52) U.S. Cl. CPC A63B 21/4045; A61B 5/1124; A61B 5/1127; G06K 9/00671; G06T 7/277; G06T 13/40; A61B 5/1128

(57) ABSTRACT

A method and system for measuring motion of a user's body part for motor rehabilitation after impairment. The system utilizes a two-dimensional optical acquisition system for detecting three-dimensional motions of at least one body part of a user for motor rehabilitation after impairment.

### CROSS REFERENCE TO RELATED APPLICATIONS

This patent application claims the benefit of U.S. Provisional Application No. 62/784,262, filed Dec. 21, 2018, entitled METHOD AND SYSTEM FOR MOTION MEASUREMENT AND REHABILITATION.

The entire content of 62/784,262 is hereby incorporated by reference.

### BACKGROUND OF THE INVENTION

### 1. Field of the Invention

The present invention relates generally to the field of rehabilitation of patients after motor impairments. More particularly, the invention relates to methods and systems for rehabilitation of patients suffering from impaired arm and hand movements due to neurological or orthopedic conditions.

### 2. Description of the Related Art

The clinical population is large, with 15 million individuals in the U.S. who experience limitation of dexterity in at least one hand. As will be disclosed below the present invention was initially designed for stroke sufferers (approximately 3 million suffer long-lasting deficits in upper extremity movements in the US due to stroke; more than 20 million worldwide), but it can also be used for patients with Parkinson's disease (about 1.5 million people), for whom intensive arm training improves arm movements. In addition, traumatic brain injury (about 5 million people) often results in upper extremity impairments, and intensive training can also be beneficial. Furthermore, a large class of orthopedic patients, such as those suffering from rotator cuff tears, frozen shoulder, etc., could benefit from this system. Shoulder pain for instance, is the number-three reason for referral to physical therapists in the US, and affects approximately 35% of the US population. Because activities of daily living often involve the upper extremities, patients who have sustained neurological or orthopedic injury need to regain functions of their arm and hand for a return to a full quality-of-life.

Motor rehabilitation often requires the patient to perform numerous correctly performed movements to be effective. Because therapists do not have the time to do this amount of training in the clinic, they give their patients “homework” exercises. Compliance is a major issue: patients have a hard time following through with their homework. They notably have difficulty staying engaged to perform high number of repetitions. In addition, they often perform the movements incorrectly, which can lead to worsened outcomes. Thus, patients ideally need an effective coach to motivate them to perform the large number of movements correctly at home.

For many conditions that affect the arm and hand, motor rehabilitation requires numerous movements to be effective. In particular, intensive task practice, in which patients actively engage in repeated attempts to produce motor behaviors beyond their present capabilities, is effective for improving upper extremity function after stroke. For instance, intensive practice consisting of 1000s of movements is effective for improving upper extremity function after stroke. Thus, the degree of recovery depends not only on the level of initial impairment but also on the amount, type, and intensity of task practice available to the patient during the recovery process.

Therapists do not have the time to provide this amount of training in the clinic, however. Thus, the number of repetitions demonstrated to be needed in research studies is in dramatic contrast with the limited time spent by the typical stroke patient undergoing neurological rehabilitation in actual therapeutic activity. For instance, for patients with stroke, only 32 movements are practiced per session on average. Therapists thus give exercise “homework” to their patients to increase the number of repetitions. However, as most people who undergo physical therapy can attest, patients have a hard time following through with homework. This difficulty to stayed engaged, together with the movements often being performed incorrectly in a home-setting environment, leads to poor outcomes.

Another problem with conventional medical rehabilitation models is that they are largely constrained by economic considerations (i.e., how many sessions the patient's health insurance will cover) and are therefore not adequate to maximize functional outcomes. Further, due to the belief that therapy is only marginally effective (which is at least in part the result of the low intensity of training, as discussed above), health insurance companies often reject requests for rehabilitation past 3 months post stroke.

In view of the shortcomings of the conventional medical practice model, there is a growing interest in employing technology for rehabilitation of upper extremity movements. The use of robotic systems for limb rehabilitation is provided such that the robot directly assists the movements of an impaired limb. Current robots retrain reach with robotic assistance. In particular, Inmotion2 (IMT, USA) (13), KINARM (BKIN, Canada), and Hocoma Power (Hocoma, Switzerland). These systems have been shown to be effective to some extent and can be used with patients that have no or little residual movement capabilities. However, these robots are mostly limited to large research and clinical centers because they are expensive, complex to maintain, and require supervision and/or assistance to use. Importantly, these systems provide motor training assisted by the robot. Outside the clinical setting, there is no robotic assistance available. Therefore, the effectiveness of conventional robots is limited.

Virtual reality (VR) systems are also known and allow users to measure and practice reaching movements. Virtual reality (VR) systems have the advantages of lower price, 3D interactions, increased safety, and can easily embed motivation principles from computer games. VR systems designed to enhance reaching movements in patients with stroke have been tested in small pilot studies. However, VR systems often require 3D goggles or projection screens to create the illusion of a 3D virtual world. Therefore, there still exists a need for effective systems and techniques to rehabilitate patients with neurological disorders such as strokes.

In partial response to the above-identified needs, the inventor of the present patent application, N. Schweighofer, was a co-inventor of U.S. Pat. No. 8,419,616, Upper Limb Measurement and Rehabilitation Method and System, issued on Apr. 16, 2013. The '616 patent discloses a method for measuring an upper limb reaching capability of a user with an impaired limb. Each upper limb is placed at a default position on a horizontal surface. A target is displayed from among a plurality of targets on the horizontal surface. One of the limbs reaches for the target. Limb choice, position information and elapsed time are sensed and recorded. The reaching limb is retracted to the default position. The displaying step through the retracting step are repeated for each of the plurality of targets, wherein each of the plurality of targets is spaced apart. The deficiencies of the '616 patent include the need to use sensors to track the motion and position of a user's arm reach. In one embodiment, a miniaturized DC magnetic tracking system is used, for example, a small (5 mm) magnetic sensor that is attached to a hand or a finger to track the reaching movements of the arm. The sensors are attached to long, thin cables such that movement of the arm is not affected by the use the sensors. The sensor cables can be taped to the user's upper extremities and are adjustable to allow a fully extended reach.

In another embodiment for a two-dimensional reaching task, the targets for reaching are LED lights embedded in the surface along with corresponding switches or buttons for a user to activate to record movement time. A switch or button is also provided at the de-fault position for recording reaction time and to ensure compliance with the measurement sequence. The surface can alternatively be formed as a capacitive touchscreen display where a user may touch the display directly to accomplish the reaching task. The display itself provides the lighted target, functions as the position sensor, and also provides instructions, user feedback and other display prompts.

In yet another embodiment for a two-dimensional reaching and grasping task, a plurality of physical targets is provided in corresponding holes in the surface and are raised up above the surface to indicate an active target for a user to reach for and grasp. These pop-up targets are physical objects having force and/or torque sensors that detect user contact and which scores a successful trial when the user applies a pre-determined amount of grasp force on the risen target. Alternatively, the targets may incorporate touch sensors to detect contact.

US Publication No. 2016/0023046A1, entitled Method and System for Analyzing a Virtual Rehabilitation Activity/Exercise, published on Jan. 18, 2016, encloses a computer-implemented method for analyzing rehabilitation activity and performance of a user during a virtual rehabilitation exercise. The system receives one of a rehabilitation activity and executed movements performed by the user during the virtual rehabilitation exercise. The rehabilitation activity defines an interactive environment to be used for generating a simulation that corresponds to the virtual rehabilitation exercise. The rehabilitation activity includes at least one virtual user-controlled element and input parameters, determining movement rules corresponding to the one of the rehabilitation activity and the rehabilitation exercise. Each one of the movement rules including a correlation between a given group consisting of at least a property of the virtual user-controlled element and a body part, and at least one of a respective elementary movement and a respective task-oriented movement. The rehabilitation activity also determines a sequence of movement events corresponding to the one of the rehabilitation activity and the executed movements. Each one of the movement events corresponds to a given state of the property of the virtual user-controlled object in the interactive environment, the given state corresponds to one of a beginning and an end of a movement, determining a movement sequence including at least one of elementary movements.

US Publication No. 2014/0371633A1, entitled Method and System for Evaluating a Patient During a Rehabilitation Exercise, published Dec. 18, 2014, discloses in accordance with a first broad aspect, a computer-implemented method for evaluating a user during a virtual-reality rehabilitation exercise, comprising: receiving a target sequence of movements comprising at least a first target elementary movement and a second target elementary movement, the first target elementary movement defined by a first body part and a first movement type and the second target elementary movement defined by a second body part and a second movement type, the first and second target elementary movements being different; receiving a measurement of a movement executed by the user while performing the rehabilitation exercise and interacting with a virtual-reality simulation comprising at least a virtual user controlled object, a characteristic of the virtual user-controlled object being controlled by the movement.

US Publication No. 2018/0193700A1, entitled Systems and Methods for Facilitating Rehabilitation and Exercise, published Jul. 12, 2018, discloses an exercise system which includes a user interface device sized and configured to fit within a user's hand, the user interface device including a microcontroller configured to control operation of the device, a first sensor configured to sense movements of the device, a second sensor configured to sense forces applied to the device, and a communication device configured to communicate data concerning the sensed movements and forces to a separate device.

U.S. Pat. No. 7,257,237B1, entitled Real Time Markerless Motion Tracking Using Linked Kinematic Chains, issued Aug. 14, 2007 discloses a markerless method for tracking the motion of a user in a three dimensional environment using a model based on linked kinematic chains. The invention tracks robotic, animal or human subjects in real-time using a single computer with multiple video cameras and does not require the use of markers or specialized clothing. A simple model of rigid linked segments is constructed of the user and tracked using three dimensional volumetric data collected by multiple video cameras. A physics-based method is then used to compute forces to align the model with subsequent volumetric data sets in real-time. The method is able to handle occlusion of segments, provides for error recovery, and accommodates joint limits, velocity constraints, and collision constraints. The method further provides for elimination of singularities in Jacobian based calculations, which has been problematic in alternative methods.

Generally, the above inventions cannot provide effective and motivating home motor training.

As will be disclosed below the present invention provides the capability of providing high doses of intensive training and minimization of compensatory movements via movement tracking with a single 2D camera.

### SUMMARY OF THE INVENTION

In one aspect, the present invention is embodied as a method and system for measuring motion of a user's body part for motor rehabilitation after impairment. The system utilizes a two-dimensional optical acquisition system for detecting three-dimensional motions of at least one body part of a user for motor rehabilitation after impairment.

In a preferred embodiment, the system utilizes motion estimations from a mechanical (kinematic) model of the body to generate an avatar through augmented reality. In some embodiments, motion estimations are provided by a Kalman filter with a mechanical model of the body. In some embodiments, an artificial intelligence system (e.g. deep learning network) is used to estimate the motion of at least one body part of a user.

### BRIEF DESCRIPTION OF THE DRAWINGS

FIG. 1A shows the system for measuring motion of a user's body part for motor rehabilitation after impairment, of the present invention, in an operating environment.

FIG. 1B illustrates an example of a screen display seen by the patient during arm movement exercise by the system of the present invention.

FIG. 1C shows an example of weekly reports generated by the system, of the present invention.

FIG. 2 shows a flow chart illustrating the overall sequence of the physical therapy exercise, through the system, of the present invention.

FIG. 3A illustrates three locations of the passive markers positioned on the patient.

FIG. 3B illustrates arm angles estimated by the present system from an iPad front camera compared to angles given by a Kinect v2 depth camera that uses an infrared depth sensor.

FIG. 4 is a flow diagram illustrating the decision point as to when a body part is tracked or not being tracked during a movement. When the body part is not being tracked, the movement of the hand is predicted by a minimum jerk model that receives 1) the state of the hand just before it was lost (i.e., when the body part was still tracked) 2) the position of the target presented.

FIG. 5 illustrates the motion estimations using a state-of-the-art Kalman filter with an upper extremity kinematic model when the upper arm marker is visible (status=1) and when the marker is lost (status=0).

FIG. 6 is a graph showing movement predictions to multiple targets with complete loss of the distal (wrist) marker using the combined minimum jerk model and the Kalman filter, compared to tracking without marker loss.

The same elements or parts throughout the figures of the drawings are designated by the same reference characters, while equivalent elements bear a prime designation.

### DETAILED DESCRIPTION OF THE INVENTION

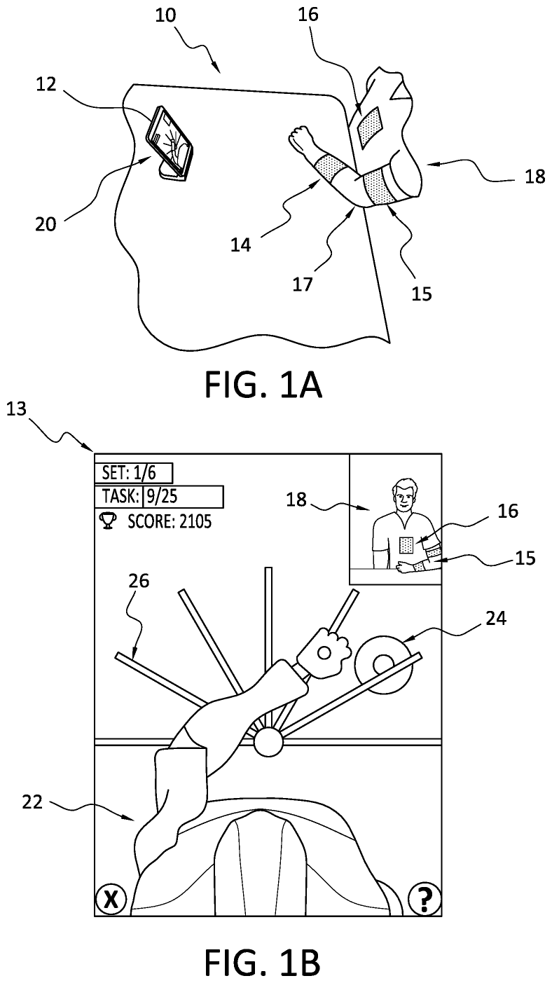

Referring now to the drawings and the characters of reference marked thereon, FIG. 1A illustrates a preferred embodiment of the system for measuring motion of a user's body part for motor rehabilitation after impairment, in accordance with the principles of the present invention, designated generally as 10. A two-dimensional optical acquisition system, typically a two-dimensional camera 12, is utilized for detecting three-dimensional motions of a user's body part(s), (e.g. the lower arm 14, the middle upper body (or the trunk) 16, the upper arm 15, and the elbow 17) for motor rehabilitation after impairment. In this illustration, the patient 18 is seated at a table, facing a mobile device 20 (phone or tablet), which is positioned vertically on the table about one meter away. The camera 12, typically the front camera 12 of the device, is viewing the patient's upper body. FIG. 1B illustrates an example of a screen display 13 seen by the patient 18 during motor rehabilitation training generated the system 10 of the present invention. In this embodiment, the patient 18 sees an avatar 22 of him/herself, which moves in real time as the patient 18 moves. Although, in this illustration the patient is shown seated at a table he could be standing for rehabilitation of the leg, gait, posture, etc. Although an avatar 22 is shown other virtual objects can be used. Furthermore, although a mobile device is shown other devices such as a PC with a webcam can be used. In other embodiments, for example, a mobile device and an external webcam (camera) can be used. The goal of this motor rehabilitation training is to catch targets 24 as quickly and as accurately as possible, following the guidance lines 26 placed on the screen display 13 by the system 10, (e.g. moving the trunk 16 or elevating the upper arm, or the shoulder 15) beyond abnormal movement parameters (such as angles or position) that deviate beyond thresholds that may be predefined, learned, and/or modified. In other embodiments of exercises, speed is not emphasized as instructions are on movement quality. In other embodiments, guidance lines 26 are not used although the guidance lines aid in providing straight lines. Audio-visual feedback of the movement speed and abnormal movements can be displayed on screen display 13 when triggered. In some embodiments, the targets are in 2D on the virtual table. In other embodiments, they could be in 3D space for reaching by moving the arm about the table. The targets could be virtual objects, moving (as in some video games). In some embodiments, reaching “through the target” is marked as a success. In some embodiments, hand tracking is implemented with the task being to grasp the targets or the objects.

FIG. 10 shows an example of weekly reports 11 generated by the system 10. These reports may include e.g. quantity of movements 19, movement speed 21, and movement quality 23. Also, these reports can be seen by the patient or the therapists, which are saved in a database in the cloud. Thus, this system uses augmented reality to motivate patients to do their physical therapy exercises at home via real-time tracking of the patient's movements on, for example, a mobile device such as a tablet or a phone. Although FIG. 1 illustrates use of upper arm exercises, the present inventive concepts can also be used with other parts of the body.

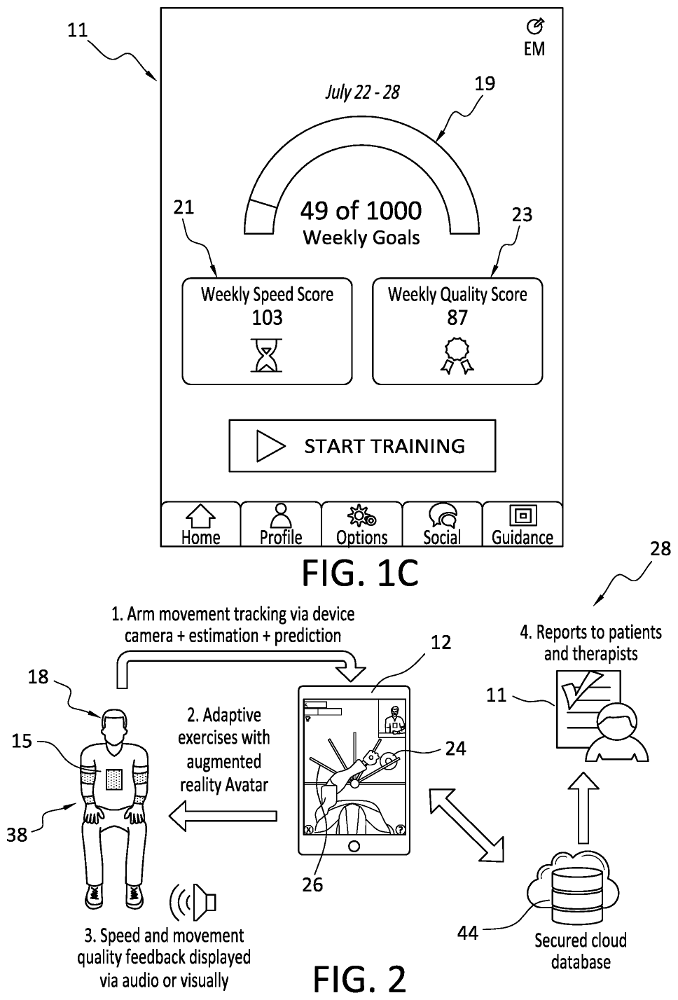

Referring now to FIG. 2, a flow chart designated generally as 28, shows the overall sequence of the physical therapy exercise, through the system 10. First, the camera 12 tracks the position of the middle upper body 15 of the patient 18, and the impaired arm 38 by tracking the positions and orientations through a human kinematic model. Such a model may be generated by an artificial intelligence system, such as a deep learning network system, and in particular a Convolutional Neural Network (ConvNet). In an example of using deep learning networks, the 2D joint position is directly predicted from images using a cascade of ConvNets that focuses on small parts of the image around the joint. A sequence of ConvNets repeatedly produces 2D (heat) maps for the location of each joint. Then, the use of confidence heatmaps computes the probability for each joint being present at every pixel, which greatly enhances the performance of 2D pose estimation. Each heatmap helps in accurately locating one 2D body joint in the image. These heatmaps are then followed by a depth regression module to generate depth information for each joint. In a preferred embodiment, an artificial intelligence system, such as ConvNets, detects three-dimensional motion of at least one body part to generate an avatar through augmented reality.

In other embodiments, physical markers may be used, as will be disclosed below in detail. In this instance, passive markers, i.e. fiducial markers, are used to calculate the coordinates (position and orientation) of body segments with respect to the camera 12. A fiducial marker or fiducial is an object placed in the field of view of an imaging system which appears in the image produced, for use as a point of reference or a measure. Such markers can be used to detect the position of the actual object, i.e. body part, which overlays a virtual object, e.g. avatar, in the system 10 with a two-dimensional camera 12. Once the camera 12 of the system 10 recognizes the body segments of the patient 18, the physical therapy exercise can begin. In one embodiment, patient 18 begins to move the impaired arm 38 to reach targets 24 by following the guidance lines 26 through the augmented reality of the system 10. The system 10 provides the patient 18 with the movement quality feedback, through comparison of current movement and estimated movement for the specific target. This enables the patient 18 to adjust his movements in order to improve his physical therapy for the impaired arm 38. The system 10 generates reports 11, that are saved onto secured cloud database 44, accessible for the patient 18 and the therapist. The reports may be, for example, weekly reports, daily reports, yearly reports.

The sizes and images of the passive markers are known by the system, often a square for instance. When the square has different size, it is at a different distance from the camera. When the marker is slanted the square is deformed and, the orientation of the marker can be computed. Thus, in one embodiment, such markers track complex arm movements.

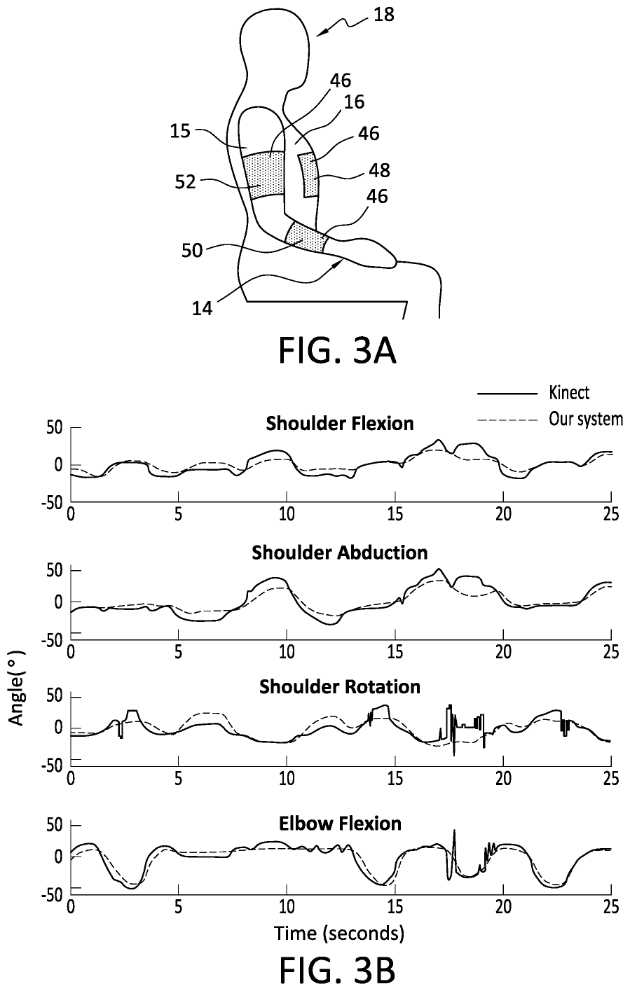

In one embodiment, three passive markers (e.g. Vuforia markers, Aruco markers, or other suitable markers) 46 may be used to calculate the coordinates (position and orientation) of body segments with respect to the camera 12. FIG. 3A illustrates three locations of the passive markers 46 positioned on the patient 18. A flat marker 48 is attached to the middle upper body (or a trunk) 16, a cylindrical marker 50 is wrapped around the lower arm 14 (or the wrist), and a second cylindrical marker 52 is wrapped around the upper arm 15. In this example, three passive markers 46 track movement of the patient's body with a medium speed up to two meters away from the camera 12. This distance is related to the specific software that is being used. Thus, different shapes of markers can be used. In this example, Vuforia markers are used. Other methods that can be used could track markers. If a system other than Vuforia is utilized an artificial intelligence system, such as a deep learning network system can be used for detecting and tracking such markers. For instance, a Convolutional Neural Network (ConvNet) with a regression module can detect the markers and track their position and orientation in space. In other embodiments, no markers are used, as discussed above.

Hardware-based wearable solutions (such as networks of accelerometers) are relatively expensive and 3D cameras are not readily available at very low cost (except for the Microsoft Kinect for arm movements and the Motion Leap for hand movements). Thus, an alternate solution is for patients to “wear” a smart phone on the upper-arm to detect movement via the device's accelerometers. While this solution can work to monitor overall activity and possibly arm use, it generally does not meet the functional requirements discussed above because of the inability to track hand trajectories due to the motion of the arm and to adequately detect abnormal movements.

Therefore, a preferred embodiment is to track whole arm movements with a 2D camera, for instance, the front camera of a mobile device, or a webcam connected to the mobile device, or to a PC. Tracking 3D movements with a 2D camera is challenging. In one embodiment, three methods have been used to solve this problem. A first method is to estimate the position of the impaired arm and of the upper body in real-time by tracking the positions and orientations of three passive fiducial markers, as noted above relative to FIG. 3A. One marker is placed on the forearm, the second on the upper arm, and the last on the upper body. Second, to robustly estimate arm movements in the face of noise and marker loss, a fine-tuned, unscented Kalman filter is preferably used that combines the marker data with predictions from a kinematic of the body model and arms. Third, because relatively long periods of marker loss can occur relatively frequently, especially for forearm movements that are oriented toward the camera, a predictive algorithm is utilized that includes target information and a minimum jerk model of the movement. Although it is preferred that an unscented Kalman filter is used other types of Kalman filter/estimation algorithms could be used, such as a simple low pass filter (or in some embodiments, no filter at all).

Referring now to FIG. 3B, graphs of arm angles vs. time for shoulder flexion, shoulder abduction, shoulder rotation and elbow flexion for the present system superimposed with the same graphs for the Kinect v2 system. With the present system, the estimates were obtained utilizing an iPad front camera. The Kinect v2 system uses a depth camera with an infrared sensor. As can be seen by the graphs, there is a very close correlation between the data using the 2D camera of the present system and the results from the Kinect v2 system. In addition, tracking with the Kinect v2 system is more noisy than tracking with a preferred embodiment of the present system.

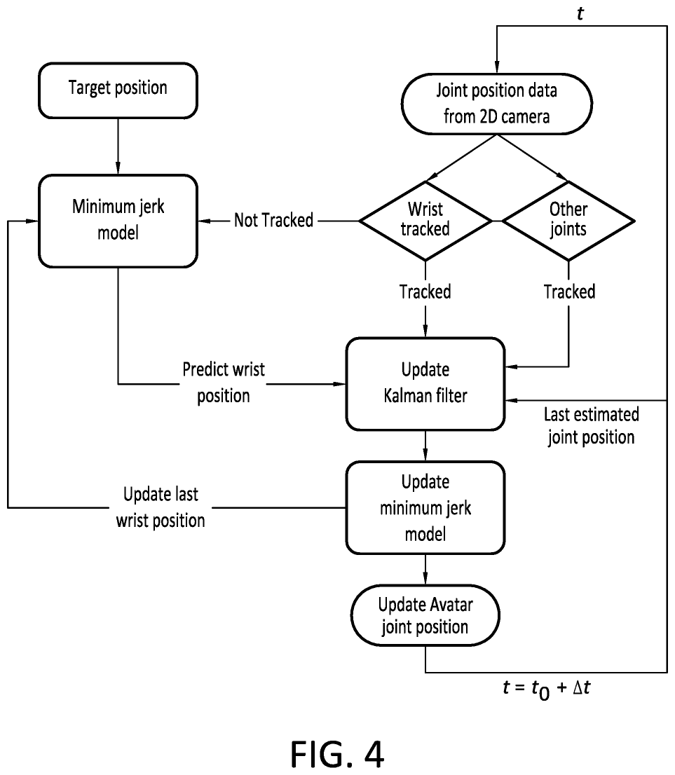

Referring now to FIG. 4, in the current time-frame, the body position data is read from the 2D camera from the markers. If the wrist position is tracked, the Kalman filter is updated with the actual wrist position. If the wrist position is not tracked, the wrist position is predicted with the minimum jerk model. Then the Kalman filter model is then updated from either actual or predicted wrist position along with other joint position data read from the camera. If other joint position data cannot be read from the camera, the Kalman filter uses previous estimates of these other joints, together with the actual or predicted wrist position, to estimate the current joint positions. The avatar position is then updated to reflect the current estimated position of the patient. In the next time-frame, the joint data is obtained from the 2D camera, and the loop continues. If the wrist is not tracked, the hand position is then predicted using the minimum jerk model.

The three methods, mentioned above, that are used to track 3D movements with a 2D camera are discussed below:

1) Movement Tracking

In one embodiment, three fiducial markers are attached to the subject's arm and body; a flat marker is attached to the middle upper body with a clip and/or Velcro, a cylindrical marker is wrapped around the forearm (around the wrist), and a second cylindrical marker wrapped around the upper arm. These fiducial markers are used to calculate the coordinates (position and orientation) of arm and upper body segments with regards to the camera. Such markers are typically used in Augmented Reality (AR) applications to detect the position of an actual object on which to overlay a virtual object. By tracking several markers attached to a person's arm and upper body in real-time, we can track the person's movements. Because each marker is simple to detect computationally, the images can be processed in real-time, even with the small CPUs of phones or tablets.

Results of tracking: testing shows that the three markers can be detected up to 2 m away from the camera in multiple orientations and in multiple lighting conditions. Initial issues with glares were resolved by using mat paint and marker material (neoprene).

Data in FIG. 3A show how the marker can track three shoulder angles (flexion, abduction, rotation) and the elbow angles during various arm movements. The trajectories are compared to those recorded from the Kinect (used in multiple rehabilitation software systems, such as Jintronix, Reflexion Health, etc), see FIG. 3B. As can be seen, tracking is of similar quality between the two systems, with less noise for the Vuforia markers.

2) Movement Estimation

Testing showed that the loss of one marker is relatively common during arm movement training. Such loss is typically due to 1) very fast movements, 2) the cylindrical upper arm marker being too slanted with regards to the line of sight of the device's camera (this happens when patients are slouching), or 3) the long-axis of the cylindrical forearm marker being nearly aligned with the line of sight of the camera (this happens, for instance, when a patient training his/her left impaired left arm makes a reaching movement from the resting position (as in FIG. 1A) to leftward targets).

Robust movement estimation algorithms that use human kinematic (“skeleton”) models can compensate, at least in part, for such marker loss because the model incorporates the position and orientation of the three markers. If detection of single marker fails, data from the other two markers are available.

An unscented Kalman filter has been utilized, which extends the work of Adams, R. J., Lichter, M. D., Krepkovich, E. T., Ellington, A., White, M., & Diamond, P. T. (2015). Assessing upper extremity motor function in practice of virtual activities of daily living. IEEE Transactions on Neural Systems and Rehabilitation Engineering, 23(2), 287-296.

The unscented Kalman filter combines marker data, when available, with prediction from a “skeleton” body model 7 segments (a spine segment, and for each left and right arm, an upper arm, a forearm, and a clavicle) with a total of 17 degrees of freedom (DOF). Each arm model has two links (proximal and distal) and contains 5 DOF: Forearm supination/pronation, elbow extension, shoulder internal/external rotations, shoulder abduction/adduction, and shoulder elevation. Each arm is mounted on a shoulder joint that can move forward/backward and up/down, via 2DOF rotation of a clavicle bone, itself connected to a “spine” link that can move in 3D space. The following constraints were added to the, a) joint rotations to anatomical limits (via virtual springs with high stiffness and non-linear dampers), and b) simple muscle dynamics.

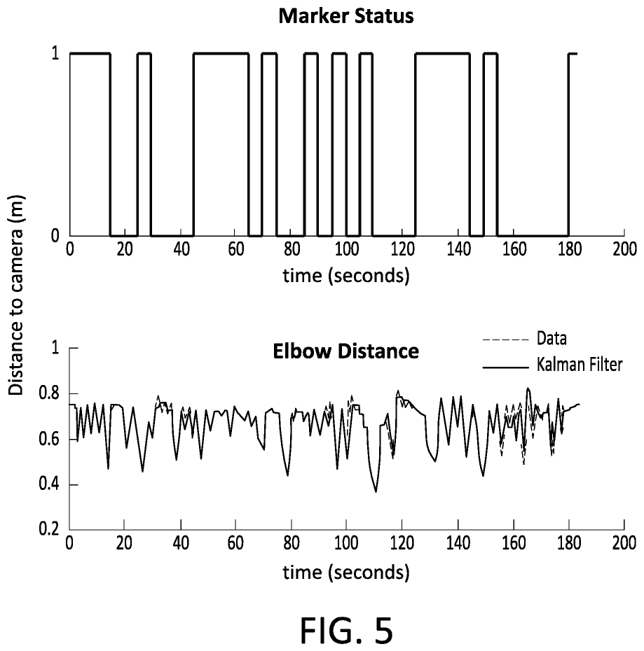

Results of estimation: To test the robustness of marker loss, a sequence of marker loss was simulated for the upper arm marker during 180 sec. In FIG. 5, actual distance from the elbow to the camera data and estimated distance when the marker is lost is shown: despite complete loss of the upper arm marker for several seconds, estimation of the distance from the elbow to the camera is very robust.

Note that simultaneous loss of both the upper and lower arm markers is catastrophic, and no estimation or predictive methods can recover from such a loss. In this case, we interrupt the exercises, and the patient hears and sees a message “please return to your resting position”—in this position, all markers are normally well detected by the camera, and the exercise can resume. Our testing shows that such simultaneous of two or more marker loss occurs rarely, about once or twice per session.

3) Movement Prediction

In addition to movement estimation, we developed a method for movement prediction. This was needed because losses of long duration (e.g., a few 100 milliseconds or more) of the distal forearm marker can lead to severe decrease in performance of the filter. This especially happens when there is little motion of the upper arm marker, and therefore little available data to update the Kalman filter when the forearm marker is lost (for instance, movements to rightward targets (for right arm training) from the home resting position, largely involve elbow flexion, and therefore yield no movements of the upper arm marker).

To solve this issue, we predicted movements at each trial by including target information as the goal of the movement in a minimum jerk model proposed by Flash, T., & Hogan, N. (1985). The coordination of arm movements: an experimentally confirmed mathematical model. Journal of neuroscience, 5(7), 1688-1703. (This is one possible algorithm for prediction of arm movements. Other ones could be used. (Minimum jerk is the simplest.)) The minimum jerk model adequately models hand trajectories in non-impaired subjects by creating realistic straight-line movements with bell-shape velocity profiles. We acknowledge that patients with impairment of upper arm movements will typically not move their hand in a straight line with a single velocity peak and will typically exhibit jerky movements. Nevertheless, because we update the predictions are every time step, and combine the minimum jerk model with the Kalman filter, which still receives inputs from the other two markers (see below for more details), our results show that this approach largely improves estimations when the distal marker is lost.

The minimum jerk model predicts the movement of the hand at time t as (here, we only show the update equation for the x position, similar equations for y and z): x(t)=x₀+(xₒ−x₁)(15 T⁶−6T⁵−10T³), where xₒ is the initial position, x_{f} the target position, and T, the normalized duration of the movement T=t/tf.

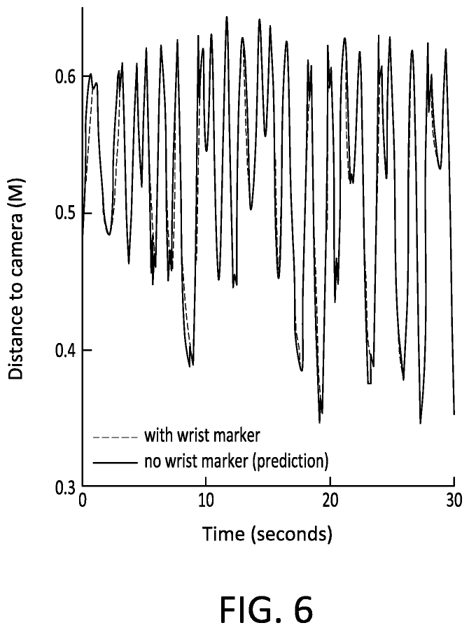

Results of predictions: When the distal forearm marker is lost, at the next step, we use position data from the two other markers as well as prediction of the wrist position from the minimum jerk model. Such a strategy largely increases the robustness of the motion tracking system in case of forearm marker loss. In FIG. 6, we performed multiple movements to different targets on a table with complete loss of the forearm marker for 30 seconds (this is a simulated loss, so we can compare the results to position estimated with all markers). As can be seen, the tracking of the wrist in the full system with minimum jerk predictions without the distal marker very closely approximate the position estimated when no markers are lost.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), General Purpose Processors (GPPs), Microcontroller Units (MCUs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software/and or firmware would be well within the skill of one skilled in the art in light of this disclosure.

In addition, those skilled in the art will appreciate that the mechanisms of some of the subject matter described herein may be capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communication link, a wireless communication link (e.g., transmitter, receiver, transmission logic, reception logic, etc.)).

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

As mentioned above, other embodiments and configurations may be devised without departing from the spirit of the invention and the scope of the appended claims. For example, although the present invention, in one example, has been described with respect to a mobile phone with only one camera, it is understood that the present inventive concepts are applicable to mobile phones with more than one 2D cameras. Furthermore, although the invention has been discussed relative to rehabilitation it can have applications for use with video games.

1. A method for measuring motion of at least one body part of a user for motor rehabilitation after impairment, comprising:
utilizing a two-dimensional optical acquisition system for detecting three-dimensional motions of at least one body part of a user for motor rehabilitation after impairment.

2. The method of claim 1, wherein said step of utilizing a two-dimensional optical acquisition system comprises utilizing motion estimations.

3. The method of claim 1, wherein said step of utilizing a two-dimensional optical acquisition system comprises utilizing motion estimations with a mechanical model of the body.

4. The method of claim 2, wherein said motion estimations are provided by a Kalman filter with a mechanical model of the body.

5. The method of claim 2, wherein said motion estimations are provided by an artificial intelligence system.

6. The method of claim 5, wherein said artificial intelligence system is used to estimate the motion of said at least one body part of a user in order to generate an avatar through augmented reality.

7. The method of claim 2, wherein said motion estimations are provided by an artificial intelligence system with a mechanical model of the body.

8. The method of claim 1, wherein said three-dimensional motion of at least one body part of the user is detected through passive fiducial markers.

9. The method of claim 8, wherein said passive fiducial markers are used to estimate the motion of at least one body part of a user in order to generate an avatar through augmented reality.

10. The method of claim 1, wherein said three-dimensional motion of at least one body part of the user is detected through passive fiducial markers in conjunction with an artificial intelligence system for detecting and tracking said passive fiducial markers.

11. The method of claim 1, wherein said step of utilizing a two-dimensional optical acquisition system comprises utilizing augmented reality to generate an avatar of a patient.

12. The method of claim 1, wherein said two-dimensional optical acquisition system is on a mobile device.

13. The method of claim 1, wherein said two-dimensional optical acquisition system is on a mobile device having more than one two-dimensional cameras.

14. A system for measuring motion of at least one body part of a user for motor rehabilitation after impairment, comprising:
a two-dimensional optical acquisition system for detecting three-dimensional motion of at least one body part of a user for motor rehabilitation after impairment.

15. The system of claim 14, wherein said two-dimensional optical acquisition system comprises utilizing motion estimations.

16. The system of claim 14, wherein said two-dimensional optical acquisition system comprises utilizing motion estimations with a mechanical model of the body.

17. The system of claim 14, wherein passive fiducial markers are utilized for said detecting three-dimensional motion of at least one body part.

18. The system of claim 14, wherein passive fiducial markers, in conjunction with an artificial intelligence system, are utilized for said detecting three-dimensional motion of said at least one body part, by tracking said passive fiducial markers.

19. The system of claim 14, wherein an artificial intelligence system is utilized for said detecting three-dimensional motion of at least one body part.

20. The system of claim 14, wherein said two-dimensional optical acquisition system comprises utilizing augmented reality to generate an avatar of a patient.
